(19) United States
(12) Patent Application Publication
(10) Pub. No.: US 2008/0262139 A1
(43) Pub. Date: Oct. 23, 2008

(54) Flame retardant crosslink agent and epoxy resin compositions free of halogen and phosphorous

(75) Inventors: Hsin-Ho Wu, Hsinchu County (TW); Weita Yang, Taoyuan County (TW); Ching Cheng Hsueh, Changhue County (TW)

Correspondence Address: BIRCH STEWART KOLASCH & BIRCH, PO BOX 747, FALLS CHURCH, VA, 22040-0747, US

(73) Assignee: Industrial Technology Research Institute

(21) Appl. No.: 11/898,499

(22) Filed: Sep. 12, 2007

(30) Foreign Application Priority Data
Apr. 23, 2007 (TW) 96114236

(51) Int. Cl. C09K 21/14; C07C 317/14; C07C 39/17; C07D 209/48; C07D 307/89; C07F 5/02; C08K 5/56

(52) U.S. Cl. 524/413; 524/425; 524/436; 524/612; 548/476; 549/240; 568/34; 568/6; 568/744

(57) ABSTRACT

Disclosed is a flame retardant crosslink agent free of halogen and phosphorous. The crosslink agent can be applied in epoxy resin composition, such that the composition achieves V0 under UL-94 standard after curing. The crosslink agent, a novolac structure modified by nitrogen-containing and/or silicon containing compound, may collocate with inorganic powder, such that the thermal cured epoxy resin composition contains thermal retardancy, low expansion, low water uptake, and high glass transfer temperature properties. The epoxy resin composition including the crosslink agent of the invention can be applied in a prepreg used in copper clad laminates or printed circuit plates.

### BACKGROUND OF THE INVENTION

1. Field of the Invention

The present invention relates to a halogen-free and phosphorous-free flame retardant epoxy resin composition, and in particular relates to a flame retardant crosslink agent thereof.

2. Description of the Related Art

Epoxy resin is widely applied in industrial use due to its electrical property, size stability, thermal resistance, chemical resistance, and adhesion. For example, epoxy resin can serve as a protective coating, an adhesive agent, packaging material for integrated circuits, or a composition. Epoxy resin plays an important role in electrical material such as copper clad laminate. In the 1960's, halo compound such as tetrabromo bisphenol A (TBBA) was introduced in epoxy resin to improve its flame retardancy. The halo compound, despite efficiently improving the flame retardancy of the epoxy resin, also released toxic chemicals such as dioxin and/or furan when burning. Therefore, the halo compound was replaced by a halo-free compound to produce halo-free flame retardants.

In U.S. Pat. Nos. 6,646,064, 6,645,631, 6,797,821, 6,291,626, 6,291,627, 6,900,269, 6,524,709, 6,645,631, and 6,645,630, phosphide such as 10-dihydro-9-oxa-10-phosphahenanthrene-10-oxide (hereinafter DOPO) and 10-(2′, 5′-dihydroxyphenyl)-9,10-dihydro-9-oxa-10-phosphahenanthrene-10-oxide (hereinafter DOPO-HQ), or derivatives thereof were used to replace the halo compound. DOPO or DOPO-HQ first reacts with epoxy resin to form a bi-functional or multi-functional epoxy resin. The formula for DOPO (Formula 1) and DOPO-HQ (Formula 2) are shown as follows:

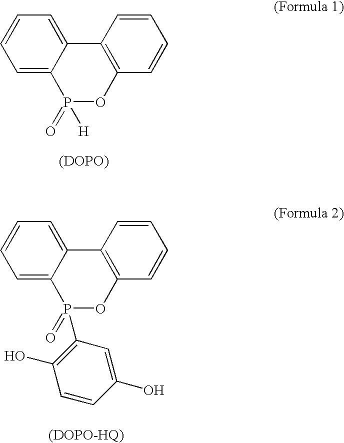

In EP Pat. No. 0384940 and 0408990, phosphide is also used to react with epoxy resin to form a bi-functional or multi-functional epoxy resin. The phosphorous-containing modified epoxy resin is of relatively higher costs, and U.S. Pat. No. 6,353,080 acknowledges this drawback. In U.S. Pat. No. 6,534,601, the bi-functional epoxy resin is first reacted with DOPO-HQ and then silicone resin to enhance flame retardancy, however, the problem of relatively higher costs still existed. In addition, adding phosphide often degrades the resin properties such as mechanical strength of water uptake, causing resin residue to pollute water resources. One solution to the aforementioned problem, is adding a high ratio of inorganic flame retardants.

Composites of organic and inorganic compounds are widely applied in several fields such as polymer nanocomposite. However, introducing large amount of inorganic flame retardants in a thermal setting polymer such as printed circuit materials will cause numerous problems. These problems include low fabricability, low flowability, low uniformity, and low post drilling yield. In JP Pat. No. 2002226558, alumina hydroxide is utilized to produce halo-free and phosphorous-free flame retardant substrates. The substrate achieves V0 level under UL-94 standard. In pressure cooker tests (PCT), heated to 121° C. and pressured to 2 atm for 2 hours, the substrate may hold 180 seconds at 260° C. However, the substrate only achieves FR-4 level for glass transfer temperature, water uptake, and solder bath resistance.

### BRIEF SUMMARY OF THE INVENTION

The invention provides a flame retardant crosslink agent, having a formula as:

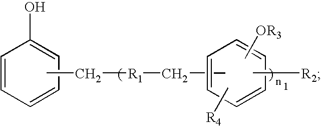

wherein n₁ is an integral from 1 to 15; R₁ is selected from phenol, phenyl, naphthol, or biphenyl; R₂ is selected from hydrogen or melamine; R₃ is selected from

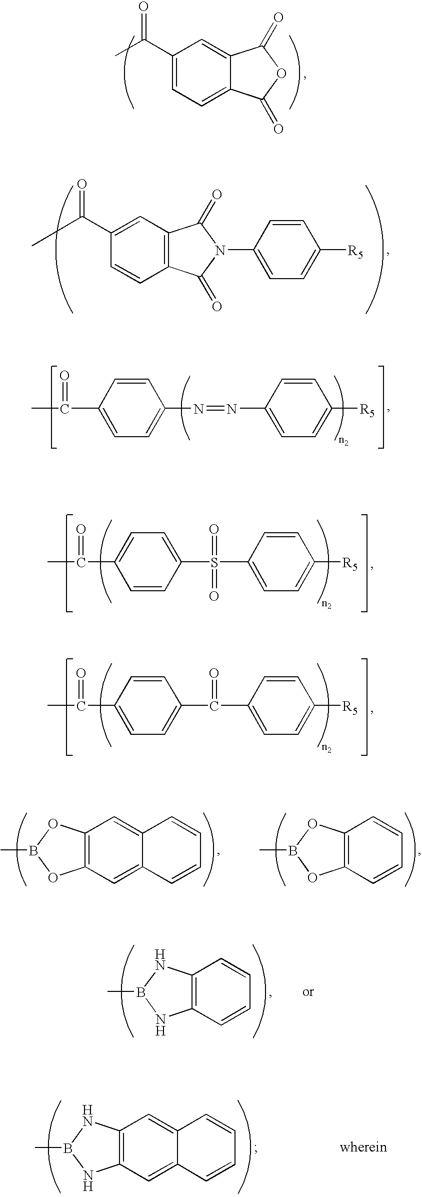

R₅ is selected from OH, COOH, CN, NO₂, OCN, or NH₂; n₂ is an integral from 1 to 3; and R₄ is

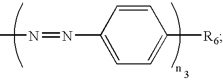

wherein R₆ is selected from OH, COOH, CN, NO₂, OCN, or NH₂; and n₃ is an integral from 1 to 3.

The invention also provides a halogen-free and phosphorous-free flame retardant epoxy resin composition, comprising 10 to 30 parts by weight of the flame retardant crosslink agent as described above, 30 to 50 parts by weight of an epoxy resin, 20 to 40 parts by weight of a curing agent, 20 to 40 parts by weight of an inorganic powder, and 0.01 to 0.1 parts by weight of catalyst.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

For overcoming the drawbacks from conventional flame retardant crosslink agents, the invention provides a flame retardant crosslink agent having a formula as formula 3.

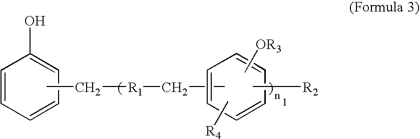

The compound having Formula 3 is a novolac resin, wherein n₁ is an integral from 1 to 15, R₁ is selected from phenol, phenyl, naphthol, or biphenyl, and R₂ is selected from hydrogen or melamine. For enhancing limited oxygen index (LOI), enhancing char formation tendency (CFT), and releasing flame retardant gas during burning, R₃ is selected from

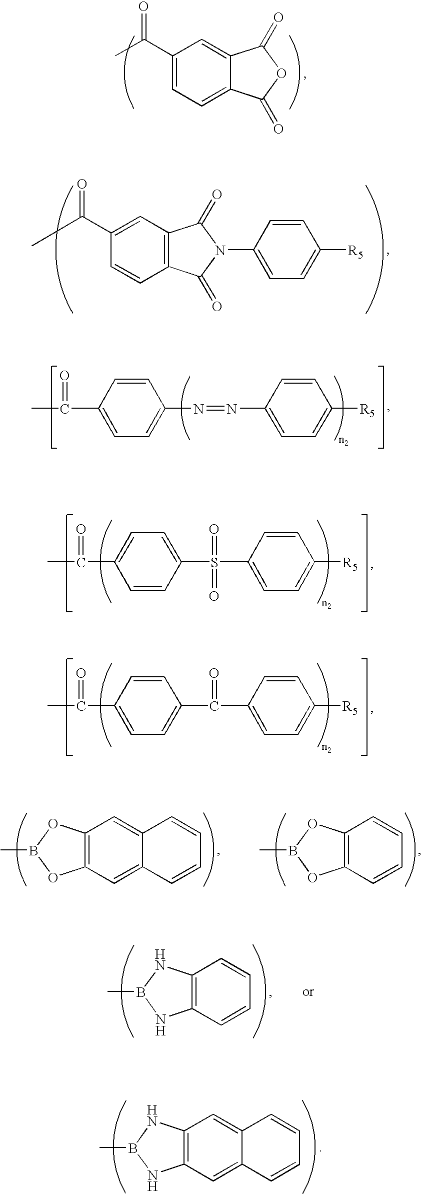

R₅ is selected from OH, COOH, CN, NO₂, OCN, or NH₂; n2 is an integral from 1 to 3. R₄ is

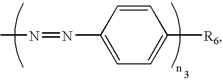

wherein R₆ is selected from OH, COOH, CN, NO₂, OCN, or NH₂; and n₃ is an integral from 1 to 3.

10 to 30 parts by weight of the flame retardant crosslink agent as described above, 30 to 50 parts by weight of an epoxy resin, 20 to 40 parts by weight of a curing agent, 20 to 40 parts by weight of an inorganic powder, and 0.01 to 0.1 parts by weight of catalyst are mixed to form a halogen-free and phosphorous-free flame retardant epoxy resin composition.

The described epoxy resin includes bisphenol A epoxy resin, bisphenol F epoxy resin, cresol novolac epoxy resin, phenol novolac epoxy resin, biphenyl-type epoxy resin, phenol p-xylene epoxy resin, phenol biphenylene resin, phenol dicyclopentadiene resin, and melamine phenol resin, or combinations thereof. At least one of the combinations thereof is novolac epoxy resin. In one embodiment, the epoxy resin includes 60 to 80 parts by weight of phenol novolac epoxy resin and 20 to 40 parts by weight of bisphenol A epoxy resin.

The described curing agent, used to increase the crosslinking degree of functional groups in the epoxy resin composition, includes phenol novolac resin, p-xylene resin, phenol biphenylene resin, phenol dicyclopentadiene resin, and melamine phenol resin, or combinations thereof. In one embodiment, the phenol groups of the curing agent and the epoxy groups of the epoxy resin have a molar ratio of 0.8 to 1.2.

The catalyst, used to accelerate the crosslinking rate, includes 2-methyl imidazole, 2-ethy-4-methyl imidazole, 2-phenyl imidazole, dimethylamino ethyl phenol, tris(dimethylaminomethyl) phenol, or benzyldimethylamine.

The inorganic powder, used to increase the flame retardancy of the epoxy resin composition, includes barium titanium oxide, silica oxide, titanium oxide, magnesium hydroxide, or zinc carbonate.

In addition to the described composition, the epoxy resin composition of the invention further includes 0.5 to 30 parts by weight of coupling agent such as amine, silane, or combinations thereof to improve the compatibility and the dispersity of the inorganic powder. The silane can be β-(3,4-epoxycyclohexyl)ethyltrimethoxysilane, γ-glycidoxypropyltrimethoxysilane, γ-aminopropyltrimethoxysilane, or N-β-(aminoethyl) γ-aminopropyltrimethoxysilane.

For conventional flame retardants, the halo material has a glass transfer temperature (T_{g}) of about 130° C., the mixture of the nitrogen-containing and phosphorous-containing material has a T_{g} of about 140 to 160° C. The epoxy resin composition of the invention has a T_{g} of about 180 to 220° C. and achieves V0 under UL-94 standard after curing. Compared to utilizing the inorganic powder as a flame retardant in the epoxy resin composition, the flame retardant crosslink agent of the invention has phenol, COOH, CN, NO₂, OCN, NH₂, and the likes to crosslink with the epoxy groups of the epoxy resin. Therefore, drawbacks such as low fabricability, low flowability, low uniformity, and low post drilling yield are mitigated. Because both, the flame retardant crosslink agent of the invention and the epoxy resin are organic compounds, the mixture between the compounds is homogeneous with out phase separation. In addition to flame retardancy and high T_{g}, the flame retardant crosslink agents of the embodiments have other advantages such as low expansion and low water uptake. Collocating with few inorganic powders, the epoxy resin composition of the embodiments may block heat transfer, reduce combustion supporting gas penetrating the composition surface, produce flame retardant gas to dilute oxygen in air, and form a char layer covering the composition surface during burning.

In one embodiment, 20 to 40 parts by weight of the inorganic powder is charged in a bottle. Acetone and 0.5 to 3.0 parts by weight of the described coupling agent are added into the bottle and evenly stirred. The described mixture, 20 to 40 parts by weight of the phenol novolac epoxy resin (NPCN-704, commercially available from Nanya Plastics Corporation), 30 to 50 parts by weight of bisphenol A epoxy resin (Epikote 828, commercially available from Shell Chemicals), phenol novolac epoxy resin served as curing agent (6000IZ, commercially available from Bakelite Corporation), 10 to 30 parts by weight of the flame retardant crosslink agent of the invention, and 0.01 to 0.1 parts by weight of imidazole served as catalyst (commercially available from Aldrich) are evenly stirred and mixed to form varnish. The varnish has a gel time of 200±10 seconds. The definition of gel time used is the period for the mixture to complete crosslinking reaction. In addition, the varnish has a pot life of 7 days.

A glass fiber cloth is then impregnated in the varnish to achieve a dipping ratio from 45% to 55%. The glass fiber cloth is then dried in a hot air circulation oven for several minutes at 170° C. to form a prepreg. In the oven, the reaction ratio of the crosslinking is controlled to be about 50%.

After stacking five prepregs, a teflon release cloth, a mirror steel plate, and a kraft are subsequently disposed on both sides of the stack. The stack is then hot pressed in a vacuum pressing machine at 200° C. for 2 hours to form a laminated sheet having a thickness of 1.0±0.05 mm. The manufacturing of a copper clad laminate is similar to that of a laminated sheet. The difference is simply the replacement of the mirror steel plates with copper foils.

### EXAMPLES AND COMPARATIVE EXAMPLES

The name, description and source of the chemicals utilized in the following Examples and Comparative Examples are listed below:

Phenol novolac epoxy resin NPCN-704 is commercially available from Nanya Plastics Corporation, bisphenol A epoxy resin Epikote 828 is commercially available from Shell Chemicals, phenol novolac epoxy resin 6000IZ served as curing agent is commercially available from Bakelite Corporation, and 2-ethyl-4-methyl imidazole served as catalyst is commercially available from Aldrich.

### Example 1

100 g of epoxy resin mixture, phenol novolac epoxy resin NPCN-704 and bisphenol A epoxy resin Epikote 828, and 30 g of the flame retardant crosslink agent of the invention were evenly mixed. The mixture was first volatilized at room temperature to remove part of the solvent, vacuum dried to remove the residue solvent, hot pressed in a vacuum pressing machine at 200° C. for 2 hours, and post baked in a hot air circulation oven at 220° C. to form a bulk material with a thickness of 1.0±0.05 mm. As shown in Table 1, the CFT% of the bulk material was measured in air and nitrogen environment at 800° C., respectively.

### Example 2

100 g of epoxy resin mixture, phenol novolac epoxy resin NPCN-704 and bisphenol A epoxy resin Epikote 828, and 50 g of flame retardant crosslink agent of the invention were evenly mixed. The mixture was first volatilized at room temperature to remove part of the solvent, vacuum dried to remove the residue solvent, hot pressed in a vacuum pressing machine at 200° C. for 2 hours, and post baked in a hot air circulation oven at 220° C. to form a bulk material with a thickness of 1.0±0.05 mm. As shown in Table 1, the CFT % of the bulk material was measured in air and nitrogen environment at 800° C., respectively.

### Example 3

100 g of epoxy resin mixture, phenol novolac epoxy resin NPCN-704 and bisphenol A epoxy resin Epikote 828, and 80 g of flame retardant crosslink agent of the invention were evenly mixed. The mixture was first volatilized at room temperature to remove part of solvent, vacuum dried to remove the residue solvent, hot pressed in a vacuum pressing machine at 200° C. for 2 hours, and post baked in a hot air circulation oven at 220° C. to form a bulk material with a thickness of 1.0±0.05 mm. As shown in Table 1, the CFT % of the bulk material was measured in air and nitrogen environment at 800° C., respectively.

### Example 4

100 g of epoxy resin mixture, phenol novolac epoxy resin NPCN-704 and bisphenol A epoxy resin Epikote 828, and 100 g of flame retardant crosslink agent of the invention were evenly mixed. The mixture was first volatilized at room temperature to remove part of solvent, vacuum dried to remove the residue solvent, hot pressed in a vacuum pressing machine at 200° C. for 2 hours, and post baked in a hot air circulation oven at 220° C. to form a bulk material with a thickness of 1.0±0.05 mm. As shown in Table 1, the CFT % of the bulk material was measured in air and nitrogen environment at 800° C., respectively.

### Comparative Example 1

100 g of epoxy resin mixture, phenol novolac epoxy resin NPCN-704 and bisphenol A epoxy resin Epikote 828, phenol novolac epoxy resin 6000IZ, and 2-ethyl-4-methyl imidazole served as catalyst were evenly mixed. The mixture was first volatilized at room temperature to remove part of solvent, vacuum dried to remove the residue solvent, hot pressed in a vacuum pressing machine at 200° C. for 2 hours, and post baked in a hot air circulation oven at 220° C. to form a bulk material with a thickness of 1.0±0.05 mm. As shown in Table 1, the CFT % of the bulk material was measured in air and nitrogen environment at 800° C., respectively.

TABLE 1

| | Comparative | Exam- | Exam- | Exam- | |
|---|---|---|---|---|---|
| | Example 1 | ple 1 | ple 2 | ple 3 | Example 4 |
| Epoxy resin | 1.0 | 1.0 | 1.0 | 1.0 | 1.0 |
| weight ratio | | | | | |
| Crosslink agent | 0 | 0.3 | 0.5 | 0.8 | 1.0 |
| weight ratio | | | | | |
| Tg | 155 | 167 | 183 | 184 | 184 |
| CFT (%, in air) | 0 | 16.3 | 25 | 28 | 30 |
| CFT (%, in | 32 | 35 | 39 | 43 | 48 |
| nitrogen) | | | | | |

As shown in Table 1, the CFT and T_{g} of the Examples 1-4 are higher than that of the Comparative Example 1. Thus, the difference is attributed to the epoxy resin composition of the invention including the flame retardant crosslink agent.

### Example 5

100 g of 4-amino bezoic acid (commercially available from Aldrich) was added to a solution of deionic water and hydrochloric acid. The acidic solution was added 53 g of sodium nitrite solution, stirred for 4 hours at 0° C. to 5° C., and then added to a mixture of 130 g phenol novolac epoxy resin 6000IZ/60 g sodium acetate (commercially available from Aldrich)/180 g ammonia solution (commercially available from Aldrich). The mixture was stirred for 4 hours at 0° C. to 5° C., terminated by adding 0. IN sulfuric acid, filtered and dried to obtain a flame retardant crosslink agent a having a yield of 90%.

100 g of epoxy resin mixture, phenol novolac epoxy resin NPCN-704 and bisphenol A epoxy resin Epikote 828, 50 g of flame retardant crosslink agent a, and 45 g of aluminum trihydroxide served as inorganic powder were evenly mixed to form varnish. The varnish has a gel time of 170±10 seconds. The definition of gel time used is the period for the mixture to complete crosslinking reaction. In addition, the varnish has a pot life of 7 days.

A glass fiber cloth is then impregnated in the varnish for about 20 minutes to achieve a dipping ratio from 45% to 55%. The glass fiber cloth is then dried in a hot air circulation oven for several minutes at 170° C. to form a prepreg. In the oven, the reaction ratio of the crosslinking is controlled to be about 50%.

After stacking five prepregs, a teflon release cloth, a mirror steel plate, and a kraft are subsequently disposed on both sides of the stack. The stack is then hot pressed in a vacuum pressing machine at 200° C. for 2 hours to form a laminated sheet having a thickness of 1.0±0.05 mm. The manufacturing of a copper clad laminate is similar to that of a laminated sheet. The difference is simply the replacement of mirror steel plates with copper foils.

As shown in Table 2, T_{g}, Z-Axis thermal expansion, flame retardation under UL-94 standard, and water uptake of the laminated sheet were measured, respectively. Water uptake (%) was measured after a 1 hour pressure cooker test (PCT).

### Example 6

125 g of 4-amino bezoic acid was added to a solution of deionic water and hydrochloric acid. The acidic solution was added 80 g of sodium nitrite solution, stirred for 4 hours at 0° C. to 5° C., and then added to a mixture of 130 g phenol novolac epoxy resin 6000IZ/60 g sodium acetate/250 g ammonia solution. The mixture was stirred for 4 hours at 0° C. to 5° C., terminated by adding 0. IN sulfuric acid, filtered and dried to obtain a flame retardant crosslink agent b having a yield of 85%.

100 g of epoxy resin mixture, phenol novolac epoxy resin NPCN-704 and bisphenol A epoxy resin Epikote 828, 50 g of flame retardant crosslink agent b, and 45 g of aluminum trihydroxide served as inorganic powder were evenly mixed to form varnish. The varnish has a gel time of 170±10 seconds. The definition of gel time used is the period for the mixture to complete crosslinking reaction. In addition, the varnish has a pot life of 7 days.

A glass fiber cloth is then impregnated in the varnish for about 20 minutes to achieve a dipping ratio from 45% to 55%. The glass fiber cloth is then dried in a hot air circulation oven for several minutes at 170° C. to form a prepreg. In the oven, the reaction ratio of the crosslinking is controlled to be about 50%.

After stacking five prepregs, a teflon release cloth, a mirror steel plate, and a kraft are subsequently disposed on both sides of the stack. The stack is then hot pressed in a vacuum pressing machine at 200° C. for 2 hours to form a laminated sheet having a thickness of 1.0±0.05 mm. The manufacturing of a copper clad laminate is similar to that of a laminated sheet. The difference is simply the replacement of mirror steel plates to copper foils.

As shown in Table 2, T_{g}, Z-Axis thermal expansion, flame retardation under UL-94 standard, and water uptake of the laminated sheet were measured, respectively. Water uptake (%) was measured after a 1 hour pressure cooker test (PCT).

### Example 7

100 g of 4-amino bezoic acid was added to a solution of deionic water and hydrochloric acid. The acidic solution was added 53 g of sodium nitrite solution, stirred for 4 hours at 0° C. to 5° C., and then added to a mixture of 85 g phenol (commercially available from Aldrich)/120 g sodium acetate/200 g ammonia solution. The mixture was stirred for 4 hours at 0° C. to 5° C., terminated by adding 0.1N sulfuric acid, filtered and dried to obtain a flame retardant crosslink agent precursor c-i having a yield of 95%.

100 g of 4-amino bezoic acid was added to a solution of deionic water and hydrochloric acid. The acidic solution was added 53 g of sodium nitrite solution, stirred for 4 hours at 0° C. to 5° C., and then added to a mixture of 130 g phenol novolac epoxy resin 6000IZ/60 g sodium acetate/180 g ammonia solution. The mixture was stirred for 4 hours at 0° C. to 5° C., terminated by adding 0.1N sulfuric acid, filtered and dried to obtain a flame retardant crosslink agent precursor c-2 having a yield of 90%.

50 g of flame retardant crosslink agent precursor c-1 and 210 g of thionyl chloride were refluxed under nitrogen for 4 hours. The residue thionyl chloride was removed by a rotavapor. The concentrate was dissolved in 200 mL THF. The solution was added a mixture of 40 g flame retardant crosslink agent precursor c-2/300 mL THF/25 g pyridine under nitrogen, stirred for 12 hours, and then added to 2L water to precipitate. The precipitation collected by filtering and drying in oven, was flame retardant crosslink agent c.

100 g of epoxy resin mixture, phenol novolac epoxy resin NPCN-704 and bisphenol A epoxy resin Epikote 828, and 50 g of flame retardant crosslink agent c were evenly mixed to form varnish. The varnish has a gel time of 170±10 seconds. The definition of gel time used is the period for the mixture to complete crosslinking reaction. In addition, the varnish has a pot life of 7 days.

A glass fiber cloth is then impregnated in the varnish for about 20 minutes to achieve a dipping ratio from 45% to 55%. The glass fiber cloth is then dried in a hot air circulation oven for several minutes at 170° C. to form a prepreg. In the oven, the reaction ratio of the crosslinking is controlled to be about 50%.

After stacking five prepregs, a teflon release cloth, a mirror steel plate, and a kraft are subsequently disposed on both sides of the stack. The stack is then hot pressed in a vacuum pressing machine at 200° C. for 2 hours to form a laminated sheet having a thickness of 1.0±0.05 mm. The manufacturing of a copper clad laminate is similar to that of a laminated sheet. The difference is simply the replacement of mirror steel plates with copper foils.

As shown in Table 2, T_{g}, Z-Axis thermal expansion, flame retardation under UL-94 standard, and water uptake of the laminated sheet were measured, respectively. Water uptake (%) was measured after a 1 hour pressure cooker test (PCT).

### Example 8

100 g of epoxy resin mixture, phenol novolac epoxy resin NPCN-704 and bisphenol A epoxy resin Epikote 828, 30 g of flame retardant crosslink agent c, and 45 g of aluminum hydroxide served as inorganic powder were evenly mixed to form varnish. The varnish has a gel time of 170±10 seconds. The definition of gel time used is the period for the mixture to complete crosslinking reaction. In addition, the varnish has a pot life of 7 days.

A glass fiber cloth is then impregnated in the varnish for about 20 minutes to achieve a dipping ratio from 45% to 55%. The glass fiber cloth is then dried in a hot air circulation oven for several minutes at 170° C. to form a prepreg. In the oven, the reaction ratio of the crosslinking is controlled to be about 50%.

After stacking five prepregs, a teflon release cloth, a mirror steel plate, and a kraft are subsequently disposed on both sides of the stack. The stack is then hot pressed in a vacuum pressing machine at 200° C. for 2 hours to form a laminated sheet having a thickness of 1.0±0.05 mm. The manufacturing of a copper clad laminate is similar to that of a laminated sheet. The difference is simply the replacement of mirror steel plates with copper foils.

As shown in Table 2, T_{g}, Z-Axis thermal expansion, flame retardation under UL-94 standard, and water uptake of the laminated sheet were measured, respectively. Water uptake (%) was measured after a 1 hour pressure cooker test (PCT).

### Comparative Example 2

100 g of epoxy resin mixture, phenol novolac epoxy resin NPCN-704 and bisphenol A epoxy resin Epikote 828, and 300 g of aluminum hydroxide served as inorganic powder were evenly mixed to form varnish. The varnish has a gel time of 170±10 seconds. The definition of gel time used is the period for the mixture to complete crosslinking reaction. In addition, the varnish has a pot life of 7 days.

A glass fiber cloth is then impregnated in the varnish for about 20 minutes to achieve a dipping ratio from 45% to 55%. The glass fiber cloth is then dried in a hot air circulation oven for several minutes at 170° C. to form a prepreg. In the oven, the reaction ratio of the crosslinking is controlled to be about 50%.

After stacking five prepregs, a teflon release cloth, a mirror steel plate, and a kraft are subsequently disposed on both sides of the stack. The stack is then hot pressed in a vacuum pressing machine at 200° C. for 2 hours to form a laminated sheet having a thickness of 1.0±0.05 mm. The manufacturing of a copper clad laminate is similar to that of a laminated sheet. The difference is simply the replacement of mirror steel plates with copper foils.

As shown in Table 2, T_{g} and flame retardancy under UL-94 standard of the laminated sheets were measured, respectively.

TABLE 2

| | Comparative | Exam- | Exam- | | |
|---|---|---|---|---|---|
| | Example 2 | ple 5 | ple 6 | Example 7 | Example 8 |
| Epoxy resin | 100 g | 100 g | 100 g | 100 g | 100 g |
| mixture | | | | | |
| Flame retardant | 0 | 50 g^{a} | 50 g^{b} | 50 g^{c} | 50 g^{c} |
| crosslink agent | | | | | |
| Inorganic | 300 g | 45 g | 45 g | 45 g | 45 g |
| powder | | | | | |
| Tg | 150° C. | 160° C. | 190° C. | 170° C. | 180° C. |
| Thermal | — | 2.5 | 2.5 | 2.9 | 2.7 |
| expansion (%) | | | | | |
| UL-94 standard | V0 | V0 | V0 | V1 | V0 |
| Water uptake | — | 0.5-0.6 | 0.2-0.3 | 0.3-0.4 | 0.4-0.5 |

^{a}diazo-benzoic acid containing crosslink agent;
^{b}sulfonyl-bipheny containing crosslink agent;
^{c}diazo containing crosslink agent.

As shown in Table 2, the amount of the inorganic powder was significantly reduced by introducing the flame retardant crosslink agent of Examples 5-7. Furthermore, the properties of the epoxy resin composition after curing, such as T_{g}, Z-Axis thermal expansion, flame retardation under UL-94 standard, and water uptake have all achieved industrial applicability.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A flame retardant crosslink agent, having a formula as: 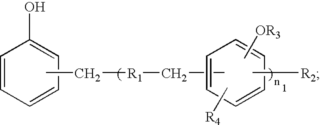
wherein n, is an integral from 1 to 15;
R₁ is selected from phenol, phenyl, naphthol, or biphenyl;
R₂ is selected from hydrogen or melamine;
R₃ is selected from 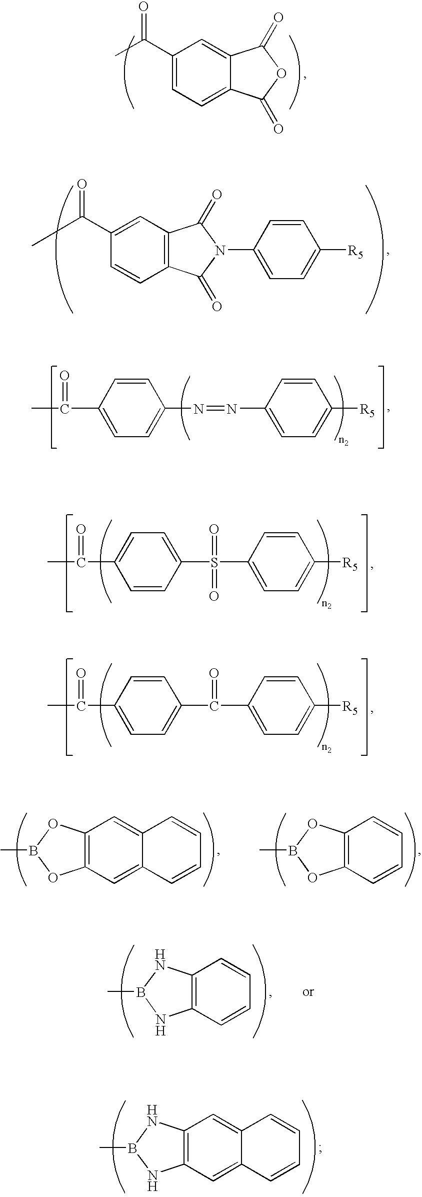
wherein R₅ is selected from OH, COOH, CN, NO₂, OCN, or NH₂; and
n₂ is an integral from 1 to 3; and
R₄ is 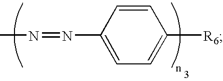
wherein R₆ is selected from OH, COOH, CN, NO₂, OCN, or NH₂; and
n₃ is an integral from 1 to 3.

2. A halogen-free and phosphorous-free flame retardant epoxy resin composition, comprising:
10 to 30 parts by weight of the flame retardant crosslink agent as claimed in claim 1;
30 to 50 parts by weight of an epoxy resin;
20 to 40 parts by weight of a curing agent;
20 to 40 parts by weight of an inorganic powder; and
0.01 to 0.1 parts by weight of catalyst.

3. The epoxy resin composition as claimed in claim 2, wherein the epoxy resin comprises bisphenol A epoxy resin, bisphenol F epoxy resin, cresol novolac epoxy resin, phenol novolac epoxy resin, biphenyl-type epoxy resin, phenol p-xylene epoxy resin, phenol biphenylene resin, phenol dicyclopentadiene resin, and melamine phenol resin, or combinations thereof, and at least one of the combinations thereof is novolac epoxy resin.

4. The epoxy resin composition as claimed in claim 2, wherein the epoxy resin comprises 60 to 80 parts by weight of phenol novolac epoxy resin and 20 to 40 parts by weight of bisphenol A epoxy resin.

5. The epoxy resin composition as claimed in claim 2, wherein the curing agent comprises phenol novolac resin, p-xylene resin, phenol biphenylene resin, phenol dicyclopentadiene resin, and melamine phenol resin, or combinations thereof.

6. The epoxy resin composition as claimed in claim 5, wherein the phenol groups of the curing agent and the epoxy groups of the epoxy resin have a molar ratio of 0.8 to 1.2.

7. The epoxy resin composition as claimed in claim 2, wherein the catalyst comprises 2-methyl imidazole, 2-ethy-4-methyl imidazole, 2-phenyl imidazole, dimethylamino ethyl phenol, tris(dimethylaminomethyl) phenol, or benzyldimethylamine.

8. The epoxy resin composition as claimed in claim 2, wherein the inorganic powder comprises barium titanium oxide, silica oxide, titanium oxide, magnesium hydroxide, or zinc carbonate.

9. The epoxy resin composition as claimed in claim 2, further comprising 0.5 to 3.0 parts by weight of coupling agent comprising amine, silane, or combinations thereof.

10. The epoxy resin composition as claimed in claim 2 has a glass transfer temperature of about 180 to 220° C. after curing.

11. The epoxy resin composition as claimed in claim 2 achieves V0 under UL-94 flame retardant standard after curing.
